(19) United States
(12) Patent Application Publication
(10) Pub. No.: US 2023/0330264 A1
(43) Pub. Date: Oct. 19, 2023

(54) COMPOSITIONS AND METHODS FOR EXOSOME-MEDIATED DELIVERY OF mRNA AGENTS

(71) Applicant: SmartCella Solutions AB, Tullinge (SE)

(72) Inventors: Kenneth CHIEN, Cambridge, MA (US); John Paul SCHELL, Sundbyberg (SE); Ran YANG, Solna (SE); Eduarde ROHNER, Stockholm (SE); Anna MARIANI, Solna (SE); Jesper SOHLMÉR, Solna (SE); Kylie FOO, Stockholm (SE); Nevin WITMAN, Watertown, MA (US)

(21) Appl. No.: 18/134,443

(22) Filed: Apr. 13, 2023

Related U.S. Application Data
(60) Provisional application No. 63/331,532, filed on Apr. 15, 2022.

(51) Int. Cl. A61K 48/00; A61K 38/46; A61K 38/18; A61K 38/17

(52) U.S. Cl. CPC A61K 48/0033; A61K 38/465; A61K 38/1866; A61K 38/1719

(57) ABSTRACT

Compositions and methods for delivery of mRNA agents to subjects are provided in which the mRNA agents are encapsulated in exosomes prepared from human stem cells or progenitor cells, such as human mesenchymal stem cells, human embryonic stem cells or human cardiac progenitor cells. The compositions and methods can be used for delivery of mRNA agents encoding therapeutics, such as enzymes (e.g., metabolic enzymes), cytokines, growth factors, antigens, antibodies or immunomodulatory agents, by administering the compositions to the subject. Methods of preparing compositions comprising exosomes encapsulating mRNA agents are also provided.

### RELATED APPLICATIONS

This application claims priority to U.S. Provisional Application No. 63/331,532, filed Apr. 15, 2022, the entire contents of which is hereby incorporated by reference.

### BACKGROUND OF THE INVENTION

The success of the COVID-19 mRNA vaccines has established mRNA agents as viable for use in humans, thus opening up a new biotechnology platform for a wide variety of prophylactic and therapeutic purposes. The current mRNA vaccines utilize modified mRNA (mmRNA) agents and incorporate the mmRNAs into lipid nanoparticles (LNPs) for delivery in vivo. While this has proved successful, there are potential limitations to this current approach.

The inherent lability of mRNA requires a delivery system to protect against degradation by nucleases and to allow cellular uptake during in vivo administration. The current approach using LNPs was first used clinically to allow the in vivo delivery of siRNA (Coelho et al. (2013) New Eng. J. Med. 369:819-829; Adams et al. (2018) New Eng. J. Med. 379:11-21). The LNPs protect the RNA cargo and are taken up via the endosomal pathway, where a portion of the RNA cargo is released from the endosome and eventually gets translated. Early versions of lipid nanoparticles containing ionizable amino lipids used to encapsulate siRNAs are described in, for example, Jayarama et al. (2012) Angew Chem. Int. Ed. Engl. 51:8529-8533. While the original versions of LNPs allowed uptake in the liver and eventually led to approval of the first siRNA therapeutic, they were associated with significant side effects (Coelho et al. (2013) New Eng. J. Med. 369:819-829; Adams et al. (2018) New Eng. J. Med. 379:11-21). However, a new generation of ionizable LNPs have been designed that lead to a larger release of the RNA cargo and marked improvement in safety and efficacy (Cheng et al. (2020) Nature Nanotech. 15:313-320). These newer versions have allowed wide-scale administration with relatively rare serious side effects, and are being harnessed to address a new wave of therapeutic candidates.

While certain improvements have been made to the LNP technology, there still are significant potential limitations, particularly with regard to repeat dosing, which often would be required in the treatment of chronic diseases with mRNA agents. Even with chemical modifications of the mRNA and packaging in more advanced lipid nanoparticles, the level of protein expression is attenuated with chronic, repeated dosing. Additionally, the inability to target mRNA delivery to specific tissues is a challenge for application to solid organ diseases. Aside from the liver where intravenous (IV) delivery of LNPs can reach much of the organ, the high efficiency, in vivo delivery of mRNA agents to other solid organs remains challenging.

Thus, while there have been various advances in the use of mRNA agents in humans, there still exists a need in the art for additional methods and approaches, in particular ones that provide alternative means than LNPs for the delivery of mRNA agents in vivo.

### SUMMARY OF THE INVENTION

The disclosure provides methods and compositions for delivery of mRNA agents in which the mRNA agents are encapsulated in extracellular vesicles (EVs), such as exosomes, derived from stem cells or progenitor cells, such as mesenchymal stem cells, embryonic stem cells, induced pluripotent stem cells and progenitor cells along various lineages, such as cardiac or pancreatic progenitor cells. The use of stem cells or progenitor cells as a source of EVs, e.g., exosomes, has advantages including the rapid growth of stem cells, allowing for preparation of large quantities of encapsulated mRNA agents, as well as the ability to control the differentiation of the stem cells and progenitor cells to thereby allow for modification of the contents of the EVs. Moreover, use of hypo-immunogenic stem cells or progenitor cells as the source of the EVs, e.g., exosomes, allows for preparation of mRNA agents encapsulated by hypo-immunogenic EVs, e.g., exosomes, which are less likely to stimulate immune responsiveness in vivo. Still further, stem cell- or progenitor cell-derived EVs, e.g., exosomes, for delivery of mRNA agents can be prepared by a variety of approaches as described herein and applied to different types of mRNA agents for a wide variety of purposes, as described herein.

Accordingly, in one aspect, the disclosure pertains to a method of delivering an mRNA agent to a subject, the method comprising administering to the subject a composition comprising extracellular vesicles, e.g., exosomes, prepared from human stem cells or human progenitor cells, wherein the EVs, e.g., exosomes, encapsulate the mRNA agent. For example, the mRNA agent can be encapsulated in the EVs by introducing the mRNA agent into the human stem cells or human progenitor cells and preparing EVs from the human stem cells or progenitor cells to thereby encapsulate the mRNA agent in the EVs.

In another aspect, the disclosure pertains to a method of preparing a composition comprising an mRNA agent, the method comprising encapsulating the mRNA agent in an extracellular vesicle, e.g., exosome, by:
(a) introducing the mRNA agent into human stem cells or human progenitor cells and preparing EVs, e.g., exosomes, from the human stem cells or progenitor cells to thereby encapsulate the mRNA agent in the EVs, e.g., exosomes; or
(b) preparing EVs, e.g., exosomes, from human stem cells or human progenitor cells and introducing the mRNA agent into the EVs, e.g., exosomes, to thereby encapsulate the mRNA agent in the EVs, e.g., exosomes.

In one embodiment, the mRNA agent is introduced into the human stem cells or human progenitor cells, or EVs, e.g., exosomes, therefrom, by electroporation. In another embodiment, the mRNA agent is introduced into the human stem cells or human progenitor cells, or EVs, e.g., exosomes, therefrom, by lipid nanoparticle-mediated transfection.

In yet another aspect, the disclosure pertains to a method of delivering an mRNA agent to a subject, the method comprising:
a) preparing a composition comprising EVs, e.g., exosomes, encapsulating the mRNA agent, wherein the composition is prepared by:
   (i) introducing the mRNA agent into human stem cells or human progenitor cells and preparing EVs, e.g., exosomes, from the human stem cells or progenitor cells to thereby encapsulate the mRNA agent in the EVs, e.g., exosomes; or
   (ii) preparing EVs, e.g., exosomes, from human stem cells or human progenitor cells and introducing the mRNA agent into the EVs, e.g., exosomes, to thereby encapsulate the mRNA agent in the EVs, e.g., exosomes; and
b) administering the composition to the subject.

In still another aspect, the disclosure pertains to a composition comprising EVs, e.g., exosomes, prepared from human stem cells or human progenitor cells, wherein the EVs, e.g., exosomes, encapsulate an mRNA agent.

In one embodiment, the mRNA agent comprises at least one modified nucleotide base. In another embodiment, the mRNA agent comprises all unmodified nucleotide bases. Various mRNA modifications are described further herein.

In one embodiment, the EVs, e.g., exosomes, are prepared from human mesenchymal stem cells (MSCs). In one embodiment, the human mesenchymal stem cells are induced mesenchymal stem cells (iMSCs). In one embodiment, the EVs, e.g., exosomes, are prepared from human embryonic stem (ES) cells. In one embodiment, the EVs, e.g., exosomes, are prepared from human induced pluripotent stem cells (iPSCs). In one embodiment, the EVs, e.g., exosomes, are prepared from human cardiac progenitor cells, such as human ventricular progenitor cells. In one embodiment, the EVs, e.g., exosomes, are prepared from human pancreatic progenitor cells, such as human β-islet progenitor cells.

In one embodiment, the stem cells from which the EVs, e.g., exosomes, are prepared are hypo-immunogenic, i.e., they have been modified to reduce their immunogenicity in a human subject. In one embodiment, the stem cells have been modified to inactivate major histocompatibility complex (MHC) Class I and/or Class II genes. In another embodiment, the stem cells have been modified to inactivate MHC Class I and/or Class II genes, as well as at least one additional gene involved in immunomodulation.

The mRNA agent can encode a therapeutic or prophylactic agent of interest for administering to the subject, e.g., based on the condition of the subject to be treated or prevented. For example, in one embodiment, the mRNA agent encodes a metabolic enzyme (e.g., for treatment of a subject with a metabolic disorder). In one embodiment, the mRNA agent encodes an antigen (e.g., for use as a vaccine in a subject). In one embodiment, the mRNA agent encodes an immunomodulatory agent (e.g., for treatment of a subject with an autoimmune disorder, cancer or other disease benefitting from immunomodulation). In various embodiments, the mRNA agent encodes an enzyme, a cytokine, a growth factor, an antigen, an antibody or an immunomodulatory protein.

The composition comprising stem cell-derived or progenitor cell-derived EVs, e.g., exosomes, encapsulating the mRNA agent can be administered to the subject by an appropriate route for the desired effect. In one embodiment, the composition is administered to an intraorgan site in the subject. In one embodiment, the intraorgan site is within the heart. In other embodiments, the intraorgan site is within the kidney, the pancreas, the liver, the lungs or the brain. In another embodiment, the composition is administered to an extravascular site in the subject. In another embodiment, the composition is administered to the subject intramuscularly. Various means for delivering the composition are described further herein.

In another aspect, the disclosure pertains to a method of delivering a functional macromolecule to cells, the method comprising:
encapsulating an mRNA encoding the functional macromolecule in extracellular vesicles (EVs) prepared from human stem cells or human progenitor cells, wherein the mRNA is, for example, at least 100 bp, 200 bp, 300 bp, 400 bp, 500 bp, 600 bp, 700 bp, 800 bp, 900 bp or 1 kilobase in length, and contacting the cells with the EVs to thereby deliver the functional macromolecule to the cells.

In other embodiments, the mRNA is at least 2 kilobases, 3 kilobases, 4 kilobases, 5 kilobases, 6 kilobases, 7 kilobases, 8 kilobases, 9 kilobases or 10 kilobases in length.

In an embodiment, the mRNA encodes a Cre recombinase. In an embodiment, the mRNA encodes a CRISPR Cas 9 protein. In other embodiments, the mRNA encodes a CRISPR Cas 12, Cas 13 or Cas 14 protein. In other embodiments, the mRNA encodes VEGF or phospholamban (PLN). In various embodiments, the mRNA agent encodes an enzyme (e.g., a metabolic enzyme), a cytokine, a growth factor, an antigen, an antibody or an immunomodulatory protein.

In an embodiment, the EVs are administered to a subject to thereby deliver the functional macromolecule to cells in vivo. In various embodiments, the EVs are exosomes, such as exosomes derived from induced mesenchymal stem cells (iMSCs).

In an embodiment, the EVs are administered to an intraorgan site in the subject, such as a site within the heart or a site within the kidney, the pancreas, the liver, the lungs or the brain. In an embodiment, the EVs are administered to an extravascular site in the subject. In an embodiment, the EVs are administered using an endoluminal delivery device.

In another aspect, the disclosure pertains to a method of expressing a protein in a cell, the method comprising:
encapsulating an mRNA agent encoding the protein in extracellular vesicles (EVs) prepared from human stem cells or human progenitor cells, wherein the mRNA agent is, for example, at least 100 bp, 200 bp, 300 bp, 400 bp, 500 bp, 600 bp, 700 bp, 800 bp, 900 bp or 1 kilobase in length; and
transfecting the cells with the EVs such that the mRNA agent expresses the protein in the cell.

In embodiments, the mRNA agent is at least 2 kilobases, 3 kilobases, 4 kilobases, 5 kilobases, 6 kilobases, 7 kilobases, 8 kilobases, 9 kilobases or 10 kilobases in length.

In embodiments, the EVs are exosomes, such as exosomes are prepared from human mesenchymal stem cells (MSCs). In embodiments, the MSCs are induced MSCs (iMSCs).

In embodiments, the mRNA agent encodes, for example, an enzyme, an antigen or an immunomodulatory protein. In embodiments, the protein encoded by the mRNA agent is, for example, Cre recombinase, CRISPR Cas 9 protein, VEGF or phospholamban (PLN).

In an embodiment, the EVs are administered to a subject to thereby deliver the protein to cells of the subject in vivo. In an embodiment, the EVs are administered to an intraorgan site in the subject. In an embodiment, the intraorgan site is within the heart. In other embodiments, the intraorgan site is within the kidney, the pancreas, the liver, the lungs or the brain. In another embodiment, the EVs are administered to an extravascular site in the subject. In an embodiment, the EVs are administered using an endoluminal delivery device.

In another aspect, the disclosure pertains to a composition comprising exosomes prepared from human induced mesenchymal stem cells (iMSCs), wherein the exosomes encapsulate an mRNA agent at least 100 bp, 200 bp, 300 bp, 400 bp, 500 bp, 600 bp, 700 bp, 800 bp, 900 bp or 1 kilobases in length. In embodiments, the mRNA agent is at least 2 kilobases, 3 kilobases, 4 kilobases, 5 kilobases, 6 kilobases, 7 kilobases, 8 kilobases, 9 kilobases or 10 kilobases in length. In an embodiment, the mRNA agent comprises at least one modified nucleotide base. In another embodiment, the mRNA agent comprises all unmodified nucleotide bases. The mRNA agent can encode, for example, an enzyme (e.g., a metabolic enzyme), a cytokine, a growth factor, an antigen, an antibody or an immunomodulatory agent. In embodiments, the mRNA agent encodes Cre recombinase, CRISPR Cas 9 protein, VEGF or phospholamban (PLN).

These and other aspects of the disclosure are described in further detail herein.

### BRIEF DESCRIPTION OF THE DRAWINGS

FIG. 1A-B are bar graphs showing in vitro expression of modified mRNA (mmRNA) by mesenchymal stem cells (MSCs) following electroporation or LNP-mediated transfection (RNAiMAX). FIG. 1A shows relative fluorescent intensity for MSCs treated with mmRNA encoding mCherry. FIG. 1B shows VEGF secretion at 24 hours, 48 hours and 72 hours for MSCs treated with mmRNA encoding VEGF.

FIG. 2 is a graph showing in vivo expression of Luciferase in mice treated with MSCs electroporated with modified mRNA (mmRNA) encoding Luciferase or mice treated with Luciferase-encoding mmRNA complexed with RNAiMAX.

FIG. 3 shows representative images showing that iMSC TSPAN markers CD9, CD63, CD81 are localized on tomographic bright-field visible intracellular/extracellular vesicles.

FIG. 4 is a bar graph showing representative flow cytometry data showing supernatant concentration of TSPAN-containing exosomes under different densities and freeze-thaw conditions.

FIG. 5 shows representative images using the Nanolive 3D Cell Explorer-Fluo to visualize transfected mRNA-594-GFP contained in tomographic bright-field visible vesicles within intracellular compartments.

FIG. 6A-6B show representative images using the Nanolive 3D Cell Explorer-Fluo to visualize transfected mRNA-594-GFP contained in tomographic bright-field visible vesicles, that are also TSPAN (CD9/63/81) positive within intracellular compartments.

FIG. 7 shows representative images using the Nanolive 3D Cell Explorer-Fluo to visualize transfected mRNA-594-GFP contained in tomographic bright-field visible vesicles, that are also TSPAN (CD9/63/81) positive within extracellular compartments.

FIG. 8 shows representative images using the Nanolive 3D Cell Explorer-Fluo to visualize translated GFP protein contained in tomographic bright-field visible vesicles, that are also TSPAN (CD9/63/81) positive within intracellular/extracellular compartments.

FIG. 9 shows representative images using standard bright-field and fluorescence microscopy to visualize translated GFP protein expressed in beating cardiomyocytes.

FIG. 10 shows representative images using the Nanolive 3D Cell Explorer-Fluo to visualize translated TSPAN-GFP protein contained in tomographic bright-field visible vesicles, while also using TSPAN (CD9/63/81) conjugated antibodies to visualize total TSPAN.

FIG. 11A-11C show representative images of recipient mouse cells following delivery of Cre mRNA using iMSC EVs. FIG. 11A shows Cre recombinase expression as detected in recipient cells using immunflourescent staining with anti-Cre antibody. FIG. 11B shows tdTomato reporter gene expression. FIG. 11C shows controls staining with DAPI.

FIG. 12 is a bar graph showing expression of Cas9 mRNA in donor cells electroporated with Cas9 mRNA and recipient cells treated with iMSC-EVs collected from donor cell supernatants.

FIG. 13. is a bar graph showing expression of CD63 in the indicated organs from mice injected under the kidney capsule with iMSCs transfected with CD63-GFP mRNA. Quantitative PCR results show the relative expression of exogenous CD63 mRNA in tissues of the indicated organs on Day 1, 3 or Day 7 in the treated group (CD63-GFP mRNA group), as compared to the control group (GFP mRNA group).

### DETAILED DESCRIPTION OF THE INVENTION

The disclosure pertains to use of extracellular vesicles (EVs), such as exosomes, derived from stem cells or progenitor cells to deliver mRNA agents to cells. In particular embodiments, the EVs, e.g., exosomes, are derived from mesenchymal stem cells, such as induced mesenchymal stem cells (iMSCs). As demonstrated in the Examples, iMSC-derived exosomes loaded with mRNA agents can be obtained by several different approaches and the loaded exosomes can be used to deliver the mRNA cargo to cells, such as beating cardiomyocytes. Furthermore, iMSCs can be modified to enhance expression of tetraspanins in the iMSCs, which thereby promotes exosome formation by the iMSCs. Cargo-loaded exosomes (e.g., iMSC-derived) can be used to deliver mRNA agents to cells, tissues, organs or bodily locations of interest, as described herein, including directly to the heart in vivo or into an extravascular space, for example using a catheter or endoluminal delivery cannula, as described herein. Local administration of a mRNA agent in vivo has been shown to allow for systemic distribution of expression of the mRNA agent.

As used herein, the term “extracellular vesicles” or “EVs” refers to lipid bilayer-encapsulated particles that are naturally released from almost all cell types yet which cannot replicate. EVs include exosomes, microvesicles and apoptotic bodies. As used herein, an “exosome” refers to a type of extracellular vesicle that is endosomally-derived and that is typically approximately 30-120 nm in size, whereas microvesicles are typically approximately 100-1000 nm in size and derived mainly from outward budding of the plasma membrane. As used herein a “loaded” EV or exosome refers to a vesicle that carries a cargo, such as an mRNA cargo, that has been introduced into the vesicle. Means for loading cargo into EVs and exosomes are described further herein. Exosomes can be detected based on detection of one or more exosome markers, non-limiting examples of which include the tetraspanin proteins CD9, CD63, CD81, CD82 and CD151.

Various aspects of the disclosure are described in the subsections below.

### I. Stem Cells and Progenitor Cells

The methods and compositions of the disclosure utilize EVs, e.g., exosomes, derived from (i.e., prepared from) stem cells or progenitor cells, e.g., human stem cells or human progenitor cells.

As used herein, the term “stem cells” is used in a broad sense and includes traditional stem cells, progenitor cells, pre-progenitor cells, reserve cells, and the like. The term “stem cell” or “progenitor” are used interchangeably herein, and refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation”.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

A. Pluripotent Stem Cells

In embodiments, a stem cell or progenitor cell used in the methods of the disclosure is pluripotent or exhibits pluripotency or a pluripotent state. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratomas formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell. The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

In one embodiment, the methods and compositions of the disclosure utilize exosomes derived from (i.e., prepared from) embryonic stem cells, e.g., human embryonic stem cells. The terms “embryonic stem cell”, “ES cell” and “ESC” are used interchangeably herein and refer to the pluripotent stem cells of the inner cell mass of the embryonic blastocyst (see e.g., U.S. Pat. Nos. 5,843,780 and 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like. In some embodiments, an ES cell can be obtained without destroying the embryo, for example, without destroying a human embryo. Numerous embryonic stem cell lines are well established and available in the art, non-limiting examples of which include ES03 cells (WiCell Research Institute) and H9 cells (Thomson, J. A. et al. (1998) Science 282:1145-1147). Culture media and culture conditions for maintaining and expanding ES cell lines are also well established and commercially available in the art. Preparation of extracellular vesicles, e.g., exosomes, from ES cells has been described in the art (see e.g., Ke et al. (2021) Stem Cell Res. & Therap. 12:21).

In one embodiment, the methods and compositions of the disclosure utilize exosomes derived from (i.e., prepared from) induced pluripotent stem cells (iPSCs), e.g., human induced pluripotent stem cells. As used herein, an “induced pluripotent stem cell” refers to a type of pluripotent stem cell that is derived from adult somatic cells but has been reprogrammed through induction of certain genes and factors to be pluripotent. Numerous human iPSC lines are well established and available in the art, non-limiting examples of which include 19-11-1, 19-9-7 or 6-9-9 cells (e.g., as described in Yu, J. et al. (2009) Science 324:797-801). Culture media and culture conditions for maintaining and expanding iPSCs are also well established and commercially available in the art. Preparation of extracellular vesicles, e.g., exosomes, from iPSCs has been described in the art (see e.g., Jeske et al. (2020) Tissue Eng. Part B: Reviews 26:129-144).

In certain embodiments, pluripotent stem cells are identified by or indicated by the expression of one or more pluripotent stem cell markers. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2.

In one embodiment, the methods and compositions of the disclosure utilize exosomes derived from (i.e., prepared from) adult stem cells, e.g., human adult stem cells. The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

B. Mesenchymal Stem Cells

In one embodiment, the methods and compositions of the disclosure utilize exosomes derived from (i.e., prepared from) mesenchymal stem cells (MSCs), such as induced mesenchymal stem cells (iMSCs) that can be prepared from pluripotent stem cells. As used herein, the term “mesenchymal stem cell” refers to multipotent adult stem cells that can self-renew by dividing and can differentiate into multiple tissues including bone, cartilage, muscle cells, fat cells and connective tissue. Mesenchymal stem cells are naturally present in multiple tissues, including umbilical cord, bone marrow, fat tissue and peripheral blood. Accordingly, in one embodiment, the MSCs used for preparation of exosomes are MSCs that have been isolated from the subject to which mRNA-loaded exosomes (prepared as described herein) are to be administered (i.e., the MSCs are isolated from the same subject to be treated with the MSC-derived exosomes).

In one embodiment, the MSCs are bone marrow mesenchymal stem cells (BMSCs), which can be directly isolated from subjects. U.S. Patent Application US2008/0279828A1 discloses methods of mobilization of bone marrow stem cells into the peripheral blood of a donor for harvesting the bone marrow stem cells, and is incorporated herein by reference in its entirety. The method comprises administering to the donor an effective amount of at least one copper chelate, to thereby expand the bone marrow stem cells in vivo, while at the same time reversibly inhibiting differentiation of the bone marrow stem cells; and harvesting the bone marrow stem cells by leukopheresis.

Alternatively, cells that differentiate into BMSCs (“BMSC precursors”) can be isolated from subjects and then exposed to one or more chemical or biological agents to differentiate into BMSCs in culture. U.S. Pat. No. 5,486,359 describes the isolation of human mesenchymal stem cells, which can differentiate into more than one tissue type (e.g. bone, cartilage, muscle, or marrow stroma) and a method for isolating, purifying, and culturally expanding human mesenchymal stem cells.

Additional sources of MSCs from adult niches including adipose/fat-derived MSCs and peripheral blood derived MSCs. In addition, MSCs from the pre/neo-natal environment can be used in the methods described herein, including umbilical and placental-derived MSCs. Human umbilical cord and placenta-derived MSCs, as well as peripheral blood derived MSCs can be isolated from patients using methods known in the art, e.g., through a combination of tissue explant cultures and/or by gradient density separation through centrifugation (Beeravolu et al. (2017) J. Vis. Exp, 122; Chong et al. (2012) J Orthop Res., 30(4):634-42. For the isolation of adipose/fat-derived MSCs, the cells can first be isolated using for example, methods involving liposuction and resection (Schneider et al. (2017) Eur. J. Med. Res. 22(1):17. Although some functional diversity exists within mesenchymal stem cells derived from different patients and/or different tissue sources, for mesenchymal stem cells to maintain their identity they should possess three functional attributes: 1) self-renewal potential; 2) ability to grow on plastics; and 3) ability to differentiate into three major cell types including osteoblast (bone), chondrocyte (cartilage) and adipocyte (fat). Additionally, regardless of the source of MSCs, the MSCs should have differentiation markers such as CD73, CD90 and the lack of CD14, CD34, and CD45 (Ullah et al. (2015) Biosci. Rep., 35(2); Fitzsimmons et al. (2018) Stem Cells Int. 2018: 8031718).

In an embodiment, the MSCs are induced MSCs (iMSCs) that have been prepared from pluripotent stem cells, such as human embryonic stem cells (ESCs) or human induced pluripotent stem cells (iPSCs). Methods of preparing iMSCs from pluripotent stem cells have been described in the art (see e.g., Soontararak et al. (2018) Stem Cells Transl. Med. 7:456-467; Yang et al. (2019) Cell Death and Disease 10:718; Xu et al. (2019) Stem Cells 37:754-765). Culture protocols for differentiation of iMSCs from pluripotent stem cells are also described in detail in U.S. Provisional Patent Application Ser. No. 63/307,368, filed Feb. 7, 2022, the entire contents of which is hereby specifically incorporated by reference.

As shown in Example 3, iMSCs express tetraspanins, such as CD9, CD63 and CD81. Tetraspanins are a protein superfamily that organize membrane microdomains, termed tetraspanin-enriched microdomains (TEMs) by forming clusters and interacting with a variety of transmembrane and cytosolic signaling proteins (see e.g., Hemler et al. (2005) Nat. Rev. Mol. Cell. Biol. 6:801-811). Since tetraspanins are expressed on various types of endocytic membranes, they have been used in the art as exosomal markers. Non-limiting examples of tetraspanins include CD9, CD63, CD81, CD82 and CD151. In an embodiment, iMSCs express at least one, and preferably a plurality (e.g., two, three, four or five) tetraspanins selected from the group consisting of CD9, CD63, CD81, CD82 and CD151. Tetraspanin expression on cells can be determined by methods well-established in the art, such as using an anti-tetraspanin antibody for immunodetection.

As shown in Example 6, transfection of iMSCs with a nucleic acid construct(s) encoding a tetraspanin(s) (e.g., mRNA encoding a tetraspanin) promotes formation of iMSC-derived exosomes that are tetraspanin positive. Accordingly, in an embodiment, iMSCs are modified (e.g., genetically engineered) to express one or more tetraspanins, such as one or more selected from the group consisting of CD9, CD63, CD81, CD82 and CD151. Regardless of whether the iMSCs endogenously express the tetraspanin(s), the cells can be modified to enhance tetraspanin expression to thereby promote exosome formation. In one embodiment, the cells are modified with one or more mRNA constructs encoding the tetraspanin(s). In one embodiment, the cells are modified with one or more DNA constructs encoding the tetraspanin(s).

C. Cardiac Progenitor Cells.

In one embodiment, the methods and compositions of the disclosure utilize exosomes derived from (i.e., prepared from) cardiac progenitor cells, e.g., human cardiac progenitor cells. The term “cardiac progenitor cell”, as used herein, refers to a progenitor cell that is committed to the cardiac lineage and that has the capacity to differentiate into all three cardiac lineage cells (cardiac muscle cells, endothelial cells and smooth muscle cells). A culture of human cardiac progenitor cells can be obtained by, for example, culturing human stem cells under conditions that bias the stem cells toward differentiation to the cardiac lineage. In certain embodiments, the stem cells that are cultured to generate human cardiac progenitor cells are human embryonic stem cells or human induced pluripotent cells. Various methods for differentiating pluripotent stem cells along the cardiac lineage to thereby generate cardiac progenitor cells are well established in the art. Moreover, preparation of extracellular vesicles, e.g., exosomes, from cardiac progenitor cells has been described in the art (see e.g., Wang et al. (2019) J. Cell Mol. Med. 23:7124-7131).

In one embodiment, the cardiac progenitor cells from which the exosomes are derived are ventricular progenitor cells, e.g., human ventricular progenitor cells. The terms “ventricular progenitor cell”, “human ventricular progenitor cell” and “HVP”, as used herein, refer to a progenitor cell that is committed to the cardiac lineage and that predominantly differentiates into cardiac ventricular muscle cells (i.e., more than 50% of the differentiated cells, preferably more than 60%, 70%, 80% or 90% of the differentiated cells, derived from the progenitor cells are cardiac ventricular muscle cells). Methods for differentiating pluripotent stem cells along the cardiac ventricular lineage to thereby generate ventricular progenitor cells are well established in the art. For examples, methods of generating human ventricular progenitors (HVPs) are described in detail in US Patent Publication Nos. 2016/0053229, 2016/0108363, 2018/0148691 and 2019/0062696. Non-limiting examples of HVP markers include ISL1, JAG1, FZD4, LIFR, FGFR3, TNFSF9, PDGFRA and NRP-1.

D. Pancreatic Progenitor Cells

In one embodiment, the methods and compositions of the disclosure utilize exosomes derived from (i.e., prepared from) pancreatic progenitor cells, e.g., human pancreatic progenitor cells. The term “pancreatic progenitor cell”, as used herein, refers to a multipotent progenitor cell originating from the developing fore-gut endoderm that has the ability to differentiate into the lineage-specific progenitors responsible for the developing pancreas, including both the endocrine and exocrine cells. A culture of human pancreatic progenitor cells can be obtained by, for example, culturing human stem cells under conditions that bias the stem cells toward differentiation to the pancreatic lineage. In certain embodiments, the stem cells that are cultured to generate human pancreatic progenitor cells are human embryonic stem cells or human induced pluripotent cells. Various methods for differentiating pluripotent stem cells along the pancreatic lineage to thereby generate pancreatic progenitor cells are well established in the art. Moreover, preparation of extracellular vesicles, e.g., exosomes, from pancreatic progenitor cells has been described in the art (see e.g., Figliolini et al. (2014) PLoS ONE 9(7):e102521; Guay et al. (2015) Cell Commun. Signal. 13:17).

In one embodiment, the pancreatic progenitor cells from which the exosomes are derived are β-islet progenitor cells, e.g., human β-islet progenitor cells. The term “β-islet progenitor cell”, as used herein, refer to a progenitor cell that is committed to the pancreatic lineage and that predominantly differentiates into pancreatic β-islet cells. β-islet progenitor cells include beta cell pro-precursor cells, which are MafB+/Pdx1+/Nkx2.2+ cells, and beta cell precursors, which express Pax1. Methods for differentiating pluripotent stem cells along the pancreatic lineage to thereby generate β-islet progenitor cells are well established in the art. For examples, methods of generating human β-islet progenitor cells are reviewed in Pagliuca and Melton (2013) Development 140:2472-2483; Zhou and Melton (2018) Nature 557:351-358; Ma et al. (2018) Proc. Natl. Acad. Sci. USA 115:3924-3929; US Patent Publication 20130344594; US Patent Publication 20150231181; US Patent Publication 20160326494; US Patent Publication 20160175363; US Patent Publication 20161777267; US Patent Publication 20161777268; US Patent Publication 20161777269; US Patent Publication 20170029778; US Patent Publication 20200199539; and US Patent Publication 202000347358.

E. Hypoimmunogenic Cells

In one embodiment, the methods and compositions of the disclosure utilize exosomes derived from (i.e., prepared from) stem cells or progenitor cells that are hypoimmunogenic. As used herein, the term “hypoimmunogenic” refers to modification of the stem cell or progenitor cells to reduce its immunogenicity in vivo (e.g., reduce it's ability to stimulate an immune response in a human subject). Typically, cells are rendered hypoimmunogenic by disabling one or more genes involved in recognition of the stem/progenitor cell by the immune system and/or activation of the immune system by the stem/progenitor cell. Genes can be disabled by standard recombinant DNA technology well-established in the art, including numerous approaches for gene “knock-out”. In one embodiment, the cells are modified to lack expression of major histocompatibility complex (MHC) genes. In one embodiment, the cells lack expression of MHC Class I and/or Class II genes. In another embodiment, the cells lack expression of one or more additional genes involved in immune recognition or activation, such as minor histocompatibility genes. In an embodiment, the cells lack expression of MHC Class I and/or Class II and also lack expression of CD47. In another embodiment, the cells lack expression of MHC Class I and/or Class II and also lack expression of CD47, PD-L1 and HLAG. Hypoimmunogenic human pluripotent stem cells, and methods of preparing them, are well known in the art (see e.g., Han et al. (2019) Proc. Natl. Acad. Sci. USA 116:10441-10446; Deuse et al. (2019) Nature 37:252-258; Deuse et al. (2019) Nature Biotechnology 37:252-258; Zhao et al. (2020) iScience 23:101162; Ye et al. (2020) Cell Prolif. 53:e12946; US Patent Publication 2019/0309259; and US Patent Publication 2021/0261916).

II. mRNA Agents

An mRNA agent used in the methods and compositions of the disclosure may be a naturally or non-naturally occurring mRNA. In one embodiment, the mRNA comprises naturally-occurring nucleobases, nucleosides or nucleotides (i.e., every nucleobase, nucleoside or nucleotide in the mRNA is naturally-occurring). In another embodiment, the mRNA includes one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA agent may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA agent may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG.

In some embodiments, the mRNA agent is an unmodified mRNA in which no chemically modified nucleosides are used but which still comprises a 5′cap structure or cap species as described above.

An mRNA agent may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA agent may include a microRNA binding site. The sequences of numerous microRNA binding sites are well known in the art.

In some embodiments, an mRNA agent comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA. In some embodiments, the modified nucleobase is a modified uracil. In some embodiments, the modified nucleobase is a modified cytosine. In some embodiments, the modified nucleobase is a modified adenine. In some embodiments, the modified nucleobase is a modified guanine. In some embodiments, an mRNA agent includes a combination of one or more of the aforementioned modified nucleobases (e.g., a combination of 2, 3 or 4 of the aforementioned modified nucleobases).

In certain embodiments, an mRNA agent is uniformly modified (i.e., fully modified, modified through-out the entire sequence) for a particular modification. For example, an mRNA can be uniformly modified with N1-methylpseudouridine (m¹ψ) or 5-methyl-cytidine (m⁵C), meaning that all uridines or all cytosine nucleosides in the mRNA sequence are replaced with N1-methylpseudouridine (m¹ψ) or 5-methyl-cytidine (m⁵C). Similarly, mRNAs agents can be uniformly modified for any type of nucleoside residue present in the sequence by replacement with a modified residue such as those set forth above.

In some embodiments, an mRNA agent is modified in a coding region (e.g., an open reading frame encoding a polypeptide). In other embodiments, an mRNA agent is modified in regions besides a coding region. For example, in some embodiments, a 5′-UTR and/or a 3′-UTR are used, wherein either or both may independently contain one or more different nucleoside modifications. In such embodiments, nucleoside modifications may also be present in the coding region.

Non-limiting examples of nucleoside modifications and combinations thereof that may be present in mmRNAs agents include, but are not limited to, those described in PCT Patent Application Publications: WO2012045075, WO2014081507, WO2014093924, WO2014164253, and WO2014159813.

In some embodiments, an mRNAs agent may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In embodiments, an mRNA agent is a “large” mRNA of at least 1 kilobase in length. In embodiments, the mRNA agent is at least 1 kilobase in length, at least 1.5 kilobases in length, at least 2 kilobases in length, at least 2.5 kilobases in length, at least 3 kilobases in length, at least 3.5 kilobases in length, at least 4 kilobases in length, at least 4.5 kilobases in length, at least 5 kilobases in length, at least 5.5 kilobases in length, at least 6 kilobases in length, at least 6.5 kilobases in length, at least 7 kilobases in length, at least 7.5 kilobases in length, at least 8 kilobases in length, at least 8.5 kilobases in length, at least 9 kilobases in length, at least 9.5 kilobases in length, or at least 10 kilobases in length. In embodiments, the large mRNA encodes a functional protein, as described herein.

mRNAs agents may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

III. Encapsulation of mRNA Agents into Extracellular Vesicles

The mRNA agent can be encapsulated into stem cell-derived EVs, e.g., exosomes, by different means, as described below. In one embodiment, the mRNA agent is introduced into the stem cell and then EVs, e.g., exosomes, are prepared from the cells. In another embodiment, EVs, e.g., exosomes, are prepared from the stem cells and then the mRNA agent is introduced into the EVs, e.g., exosomes.

A. Introduction of mRNA Agents into Cells

In an embodiment, mRNA agents are introduced into stem cells or progenitor cells and then EVs, e.g., exosomes, are prepared from the mRNA-loaded cells such that the EVs, e.g., exosomes, encapsulate the mRNA loaded within the cells.

The mRNA agents can be introduced into stem cells or progenitor cells by methods known in the art. Methods include, but are not limited to, electroporation, transfection (e.g., methods using cationic-lipid transfection reagents), and lipid nanoparticle encapsulating the mRNA agent. Loading of mRNA agents into stem cells is also described in detail in Example 1.

In a non-limiting exemplary embodiment, an mRNA agent is introduced into stem cells or progenitor cells (e.g., MSCs) using lipid-mediated transfection. For example, bone marrow-derived mesenchymal stem cells (BMSCs) are grown in culture (e.g., seeded at 20-30×10⁴ cells/well in 6-well plates or flasks) and seeded at approximately 4,000-6,000 cells per cm² in 0.2-0.4 mL/cm² media. For example, MSCs grown in T-75 flasks are generally seeded at 300,000 cells/flask in 15 mL of media. An exemplary mRNA agent is modified mRNA (mmRNA), wherein mmRNA complexes are formed with a cationic-lipid transfection reagent and incubated with the BMSCs in culture. For example, the mmRNA complexes can be, for example, formed by using 2.5 μl Lipofectamine™ MessengerMAX™ Reagent (RNAiMax Reagent and Lipofectamine 2,000 Reagent and 3,000 Reagent are also effective) per 1 μg mmRNA. Calculations are performed to transfect BMSCs at a dose of 10 pg/cell mRNA (e.g., a reporter mRNA encoding Luciferase, GFP or mCherry). Ratios of modified mRNA to cells can range from 1 pg/cell to 100 pg/cell.

In another non-limiting exemplary embodiment, an mRNA agent is introduced into stem cells or progenitor cells (e.g., MSCs) using electroporation. For example, MSCs can be grown in culture and electroporated with specific doses of the mRNA agent, e.g., mmRNA. In some embodiments, human MSCs, e.g., hBMSCs, are transfected with the mRNA agent using the Nucleofector™ 2b device and the hMSC Nucleofector™ kit (Lonza) according to the manufacturer's instructions. In brief, cells are resuspended in 100 μL Nucleofector™ solution, mixed with modified mRNA (e.g., at 100 ng-100 μg per 1 million cells), transferred to a cuvette, and electroporated using program U-23 of the Nucleofector™ device. Nucleofected samples can be placed in pre-warmed medium to recover or resuspended in a low glucose DMEM solution supplemented with FBS and Pen/strep (such as Lonza hMSC-GM™) and 10% DMSO, and frozen at −80° C. (and can be stored in liquid nitrogen tanks at −180° C.) until further use.

Following introduction of the mRNA agents into the stem cells or progenitor cells, exosomes are then prepared from the cells to thereby obtain mRNA-loaded exosomes. As used herein, the term “exosome” refers to small endosome-derived lipid particles (typically 30-120 nm in diameter) that are actively secreted by exocytosis in most living cells. Thus, exosomes naturally secreted from the stem cells or progenitor cells in culture. Accordingly, the initial step in exosome preparation is collection of culture supernatant from the stem cells or progenitor cells loaded with the mRNA agent. Supernatants (also referred to as conditioned media) can be collected, for example, daily, every two days, every three days, every four days, every five days, every six days or weekly. The conditioned media is pre-cleared of dead cells and cellular debris, typically by differential centrifugation, and then is subjected to further processing to collect exosomes.

For example, in one embodiment, the pre-cleared culture media is subjected to ultracentrifugation onto a sucrose cushion, followed by a washing step, to collect the exosomes (e.g., as described in Faruqu et al. (2018) J. Vis. Exp. 142:10.3791). Alternative methods known in the art for collecting exosomes include micro-filtration centrifugation, gradient centrifugation and size-exclusion chromatography. The recovered exosomes can be further analyzed, e.g., for yield, morphology and exosomal marker expression. Suitable methodologies known in the art for analyzing exosomes include nanoparticle tracking analysis, protein quantification, electron microscopy and flow cytometry. Various methods for isolation and analyzed exosomes are reviewed in Doyle and Wang (2019) Cells 8:727 and in Familtseva et al. (2019) Mol. Cell. Biochem. 459:1-6.

B. Introduction of mRNA Agents into Exosomes

In an embodiment, exosomes are prepared from stem cells or progenitor cells and then mRNA agents are introduced into the EVs, e.g., exosomes, such that the EVs, e.g., exosomes, encapsulate the mRNA agents.

EVs, e.g., exosomes, first are prepared from stem cells or progenitor cells as described above in subsection IIIA (except the cells are not already loaded with the mRNA agent). The EVs, e.g., exosomes, thus obtained are then used for mRNA loading, as follows.

The mRNA agents can be introduced into EVs, e.g., exosomes, by methods known in the art. Methods include, but are not limited to, electroporation, transfection and cellular nanoporation.

Introduction of nucleic acids into EVs, e.g., exosomes, has been described in the art. In one embodiment, the mRNA agent is introduced into the EVs, e.g., exosomes, by lipid-mediated transfection, such as using lipofectamine. In one embodiment, the mRNA agent is introduced into the EVs, e.g., exosomes, by calcium chloride-mediated transfection (e.g., as described in Zhang et al. (2017) Am. J. Physiol. Lung 312:L110-L121). In one embodiment, the mRNA agent is introduced into the EVs, e.g., exosomes, by cellular nanoporation (e.g., as described in Yang et al. (2019) Nature Biomed. Eng. 4:69-83). In one embodiment, the mRNA agent is introduced into the EVs, e.g., exosomes, by electroporation, e.g., using the Nucleofector™ 2b device (Lonza). In one embodiment, the mRNA agent is introduced into the EVs, e.g., exosomes, using a commercially available kit for transfection of EVs, e.g., exosomes, such as the Exo-Fect™ Exosome Transfection Kit (System Biosciences Inc.). Additional descriptions of methods for introducing nucleic acids into EVs, e.g., exosomes, are available in the art, non-limiting examples of which include Lamichhane et al. (2015) Mol. Pharmaceutics 12(10):3650-3657; Usman et al. (2018) Nature Commun. 9:2359; Yang et al. (2019) Nature Biomed. Eng. 4:69-83; and Piffoux et al. (2021) Adv. Drug Deliv. Rev. 178:113972.

### IV. Extracellular Vesicle Delivery

The composition comprising EVs, e.g., exosomes, loaded with the mRNA agent can be delivered to a subject by a means that delivers the composition to its desired location in vivo. Non-limiting examples of routes of administration for the composition include parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly.

In an embodiment, a composition is administered locally. In an embodiment, a composition is administered systemically. In an embodiment, a composition is administered by intra-organ delivery.

In some embodiments, a composition is administered directly into a solid organ (intra-organ delivery). Non-limiting examples of organs to which a composition can be directly delivered include heart, kidney, liver, pancreas, stomach spleen, lung, brain, bladder and uterus. The composition can be administered by suitable means for the route administration. In an embodiment, the composition can be administered by injection using a syringe, such as for intramuscular injection, intravenous injection or intra-arterial injection. In an embodiment, the composition is administered using a catheter or an endoluminal delivery cannula, such as for intraorgan delivery or delivery to an extravascular site.

Suitable endoluminal delivery cannulas are generally described in, for example, PCT Publication WO 2009/124990, PCT Publication WO 2012/004165, EP Patent 2291213B and U.S. Pat. No. 8,876,792, as well as Grankvist et al. (2019) J. Int. Med. 285:398-406, the contents of each of which is hereby specifically incorporated by reference.

Additionally, an endoluminal delivery device, referred to as an “Extroducer”, is described in detail in U.S. Provisional Patent Application Ser. No. 63/216,348, filed Jun. 29, 2021, the entire contents of which is hereby specifically incorporated by reference. Use of the Extroducer for in vivo delivery of a composition directly into swine heart, as compared to use of a 26G needle, is described in further detail in Example 2.

In an embodiment, the EVs, e.g., exosomes, are delivered to cardiomyocytes. As demonstrated in Example 5, iMSC-derived exosomes can effectively deliver mRNA cargo to beating cardiomyocytes. In an embodiment, the EVs, e.g., exosomes, are delivered to cardiomyocytes in vitro. In an embodiment, the EVs, e.g., exosomes, are delivered to cardiomyocytes in vivo. In an embodiment, the EVs, e.g., exosomes, are delivered to cardiomyocytes in vivo by delivery to the heart using a catheter or endoluminal delivery cannula, such as described above.

### V. Uses

The mRNA-loaded exosome compositions of the disclosure can be used for a variety of prophylactic and/or therapeutic purposes. The particular mRNA agent is selected based on the needs of the subject to be treated.

As demonstrated in Example 7, the iMSC-derived EVs, e.g., exosomes, of the disclosure can be used to deliver large mRNAs (e.g., 1 kb or greater) encoding functional macromolecules to recipient cells. In embodiments, the mRNA is at least 1 kilobase in length, at least 1.5 kilobases in length, at least 2 kilobases in length, at least 2.5 kilobases in length, at least 3 kilobases in length, at least 3.5 kilobases in length, at least 4 kilobases in length, at least 4.5 kilobases in length, at least 5 kilobases in length, at least 5.5 kilobases in length, at least 6 kilobases in length, at least 6.5 kilobases in length, at least 7 kilobases in length, at least 7.5 kilobases in length, at least 8 kilobases in length, at least 8.5 kilobases in length, at least 9 kilobases in length, at least 9.5 kilobases in length, or at least 10 kilobases in length.

Also as demonstrated in Example 7, the iMSC-derived EVs, e.g., exosomes, of the disclosure can be used to deliver mRNAs to recipient cells for a long duration, e.g., for at least 1 day, at least 2 days, at least 3 days or more. The ability for the mRNA delivered by the EVs, e.g., exosomes, to be retained for a long duration allows the possibility of using the system for delivery of mRNAs in the treatment of chronic disorders (e.g., enzyme deficiency disorders, chronic autoimmune disorders and the like) by enabling ongoing delivery of the therapeutic agent.

In one embodiment, the mRNA agent encodes as antigen and the mRNA-loaded exosomes can be used to induce an immune response to the antigen in the subject (e.g., for vaccination). In various embodiments, the antigen is from a pathogen, such as a bacteria, a virus, a yeast, a parasite or a fungus.

In one embodiment, the mRNA agent encodes an antibody (e.g., a therapeutic antibody) and the mRNA-loaded exosomes can be used for immunotherapy in any clinical situation in which therapeutic antibodies have shown to be beneficial (e.g., autoimmune diseases, cancer). Non-limiting examples of antibodies include monoclonal antibodies, human and humanized antibodies, bispecific antibodies, intrabodies and related agents that comprise immunoglobulin VH and VL regions, or binding portions thereof, for binding a target.

In one embodiment, the mRNA agent encodes an enzyme, such as an enzyme that is lacking in a lysosomal storage disorder to thereby reconstitute the enzyme in the subject. For example, in one embodiment, the mRNA agent can encode alpha-galactosidase (aGAL) in the treatment of Fabry disease. In another embodiment, the mRNA can encode N-sulfoglucosamine sulfohydrolase in the treatment of Sanfilippo A disease. In another embodiment, the mRNA can encode glucocerebrosidase in the treatment of Gaucher disease.

In one embodiment, the mRNA agent encodes a growth factor. Numerous growth factors are known in the art having well-described biological functions, non-limiting examples of which include vascular endothelial growth factor (VEGF), insulin-like growth factor (IGF), platelet-derived growth factor (PDGF), epidermal growth factor (EGF), and the like.

In one embodiment, the mRNA agent encodes a factor involved in bone development for the treatment of bone defects. Non-healing bone defects can develop following severe trauma, nonunion fractures, tumor resection or craniomaxillofacial surgery. For example, in one embodiment, the mRNA agent(s) encodes vascular endothelial growth factor (VEGF) and/or bone morphogenic protein (BMP) for the treatment of bone defects.

In one embodiment, the mRNA agent encodes an immunomodulatory agent, such as a cytokine, chemokine or immune checkpoint modulator, for purposes of immunomodulation in the subject. In one embodiment, the mRNA agent stimulates immunoresponsiveness in the subject, e.g., for use in cancer treatment. In one embodiment, the mRNA agent inhibits immunoresponsiveness in the subject, e.g., for use in autoimmune disorder treatment.

In one embodiment, the mRNA agent encodes a cardiac-related agent for use in the treatment of cardiac disorders. In such clinical situations, cardiac progenitor cells (e.g., HVPs) can be used as the source of exosomes.

In one embodiment, the mRNA agent encodes a pancreatic-related agent for use in the treatment of pancreatic disorders. In such clinical situations, pancreatic progenitor cells (e.g., β-islet progenitors) can be used as the source of exosomes.

In one embodiment, the mRNA agent encodes a functional macromolecule involved in gene modification, such as gene editing. In one embodiment, the mRNA encodes a Cre recombinase, e.g., to thereby use the delivery system of the disclosure with the Cre-Lox system. In another embodiment, the mRNA encodes a CRISPR Cas molecule, e.g., to thereby use the delivery system of the disclosure with the CRISPR gene editing system. In one embodiment, the mRNA encodes a Cas9 molecule. In other embodiments, the mRNA encodes a Cas molecule selected from the group consisting of Cas12, Cas13, Cas 14, and subtypes thereof. The CRISPR gene editing system can be used, for example, in the correcting/editing of disease-causing mutations, in the knock down of toxic gene mutations, in the interruption of tumor-specific genes and the like.

In certain embodiments, the mRNA delivered by the iMSC-derived EVs, e.g., exosomes, of the disclosure is used in the treatment of a specific disease or disorder. In an embodiment, the disease or disorder is a cardiac disease or disorder. In one embodiment, the cardiac disease or disorder is ischemia-related heart failure, such as post-myocardial infarction cardiac dysfunction. In such embodiments, the delivered mRNA can be, for example, any or all of the isoforms stemming from VEGF-A, VEGF-B, VEGF-C, VEGF-D, PlGF (hereafter referred to as the “VEGF family”), and/or HIF1α, HIF2α, HIF3α, and HIF1β (hereafter referred to as the “HIF1 family”).

In another embodiment, the disease is cardiomyopathic stemming from a genetic mutation, such as phospholamban mutation (i.e., R14del) resulting in dilated cardiomyopathy and fibrosis. In such embodiments, the delivered mRNA can encode, for example, wild-type phospholamban (PLN), a VEGF family member(s) and/or an HIF1 family member(s) and/or gene editing endonucleases, e.g., CRISPR/Cas9 (including guide RNAs) and/or base-editing endonucleases, e.g., CRISPR/Cas13 (including guide RNAs and deaminase enzymes).

In another embodiment, the disease is a skin ulcer including a diabetic ulcer. In such embodiments, the delivered mRNA can be, for example, a VEGF family member(s) and/or an HIF1 family member(s) and/or an epidermal growth factor (hereafter referred to as EGF).

In another embodiment, the disease is peripheral vascular disease (PVD). In such embodiments, the delivered mRNA can be, for example, a VEGF family member(s) and/or an HIF1 family member(s) and/or an EGF.

In another embodiment, the disease is critical limb ischemia (CLI). In such embodiments, the delivered mRNA can be, for example, a VEGF family member(s) and/or an HIF1 family member(s) and/or an EGF.

In another embodiment, the disease is a respiratory disorder such as pulmonary arterial hypertension (PAH). In such embodiments, the delivered mRNA can be, for example, a VEGF family member(s) and/or an HIF1 family member(s) and/or an angiotensin converting enzyme(s), such angiotensin I, angiotensin II, angiotensin III, angiotensin IV (known collectively hereafter as “ACE family”), and/or endothelial nitric oxide synthase 3 (known hereafter as “eNOS”).

In another embodiment, the disease is a pneumopathy, such as a pneumopathy triggered by COVID19 infection. In such embodiments, the delivered mRNA can be, for example, a VEGF family member(s) and/or an HIF1 family member(s) and/or an ACE family member(s), and/or eNOS.

### EXAMPLES

Below are examples of specific embodiments for carrying out the present invention. The examples are offered for illustrative purposes only and are not intended to limit the scope of the present invention.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

Unless otherwise stated, all reagents and chemicals were obtained from commercial sources and used without further purification.

### Example 1: Loading of Stem Cells with mRNA Agents

In this example, various types of stem cells (embryonic stem cells and mesenchymal stem cells) were loaded with an mRNA agent as a cargo and the level of protein expression from the mRNA in vitro and in vivo was compared to an equivalent mRNA agent loaded into cells using a lipid nanoparticle (LNP).

In a first set of experiments, modified mRNA (mmRNA) encoding the fluorescent protein mCherry was loaded into mesenchymal stem cells (MSCs) either by electroporation or by transfection using the Lipofectamine™ RNAiMAX reagent (ThermoFisher Scientific), an LNP reagent. The mmRNA dosage used was 5 ug per one million MSCs.

Electroporation of mmRNA into MSCs was performed using the Lonza Nucleofector 2b device. The mmRNA loading into MSCs via electroporation was performed as follows: The Supplemented Nucleofector solution was pre-warmed at room temperature. While cells were plated, media was removed and cells were washed with PBS. Cells were harvested using Lonza Trypsin solution. Trypsin was deactivated using MSC growth media. After this point, only MSC-basal media was used, as it's been suggested media containing growth serums can negatively interfere with electroporation efficiency. After cell counting, cells were placed into 15 mL falcon tubes at a ratio of 1e\hat{ }6 cells per mL media and pelleted. To perform nucleofection: Following centrifugation, supernatant was carefully and completely removed. The cells were resuspended in RT Nucleofector Solution (Lonza Nucleofector kit) at a ratio of 100 uL per 1e\hat{ }6 cells. The Nucleofector solution contained 82 uL Tube A (from the kit), 18 uL Tube B (from the kit) and desired amount of mmRNA. The mmRNA was kept on ice and only added to Nuclefector solution just prior to loading in cuvettes for electroporation. mmRNA can be concentrated to approx 1 ug/uL. Exceeding 30 uL mmRNA (total electroporation volume over 130 uL) can negatively effect transfection efficiency. Optimal total nucleofection volumes for electroporation were around 105-110 uL. In addition, the cell-mRNA mixture was not left in the Nucleofection solution for time periods longer then 15 mins, since this can also effect cell viability and mRNA integrity.

The cell—Nucleofection solution was transferred into a cuvette (supplied with Lonza's nucleofection kit), with minimal air bubbles and the selected program (U23) was applied. Immediately, 500 uL of pre-equilibrated culture media (MSC-GM) was added to every 1e\hat{ }6 hMSCs and left to equilibrate in 37° C. incubator 5-30 mins. Following nucleofection and recovery, the cells were re-counted and frozen in freeze medium or re-seeded on previously warmed culture plates containing MSC growth media. To freeze cells for later use, cells were resuspended in freeze media consisting of 90% MSC growth media and 10% DMSO, and were stored in cryovials up to 1006 per 1 mL cryo-vial.

Alternatively, transfection of MSCs with mmRNA was performed using RNAiMAX, according to the manufacturer's instructions. Expression of mCherry by the cells was assessed by standard flow cytometry.

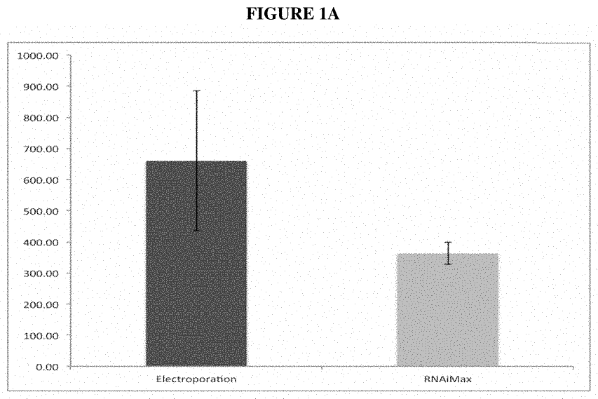

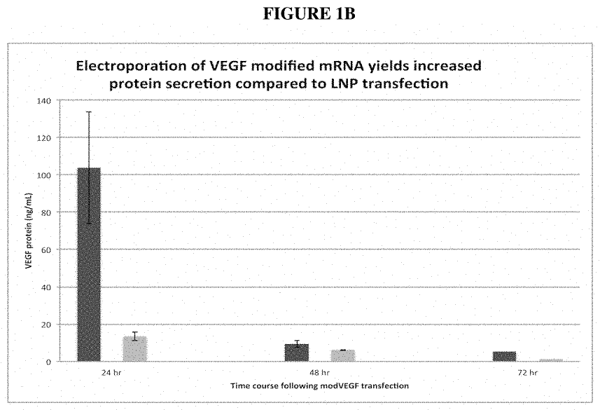

Representative results are shown in FIG. 1A, which demonstrates that electroporation of the mmRNA into the MSCs led to significantly higher protein expression as compared to LNP-mediated transfection.

In a second set of experiments, modified mRNA (mmRNA) encoding vascular endothelial growth factor (VEGF) was loaded into MSCs either by electroporation or by transfection using the Lipofectamine™ RNAiMAX reagent, as described above. The mmRNA dosage used was 20 ug per one million MSCs and cells were seeded at 50,000 cells/well. VEGF protein expression (in ng/mL) in the supernatant was assessed at 24, 48 and 72 hours post-loading. Representative results are shown in FIG. 1B, which demonstrates that electroporation of the mmRNA into the MSCs led to significantly higher protein secretion as compared to LNP-mediated transfection.

Similar electroporation experiments were also performed with unmodified mRNA encoding VEGF using bone-marrow derived MSCs, which demonstrated that unmodified mRNA also can be effectively introduced into MSCs by electroporation (data not shown).

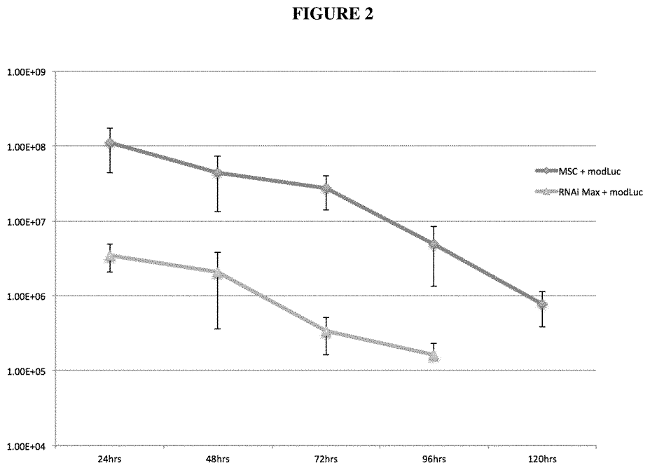

In a third set of experiments, modified mRNA (mmRNA) encoding luciferase was electroporated into MSCs at a dosage of 20 ug per one million MSCs using the Lonza Nucleofector™ technology, followed by freezing of the cells for preservation. The frozen electroporated MSCs were then thawed on the day of use. The thawed electroporated MSCs were delivered beneath the kidney capsule of mice by syringe-injection using a 27G needle. For comparison, modified luciferase mRNA was coupled with LNPs (RNAiMAX) and the mmRNA-LNP complexes were similarly delivered beneath the kidney capsule. Luciferase expression in vivo was assessed over a five day time course. Representative results are shown in FIG. 2, which demonstrates that electroporation of the mmRNA into the MSCs in vitro, followed by injection of the mmRNA-loaded MSCs in vivo, led to significantly higher protein expression in vivo compared to LNP-mediated delivery of the mmRNA in vivo.

In a fourth set of experiments, modified mRNA (mmRNA; 5 ug) encoding mCherry was loaded into human embryonic stem (ES) cells either by electroporation or by LNP-mediated transfection using RNAiMAX, as described above. Protein expression was assessed by standard fluoresecence, which demonstrated that both electroporation and LNP-mediated transfection led to efficient and comparable expression of mCherry in the human ES cells (data not shown).

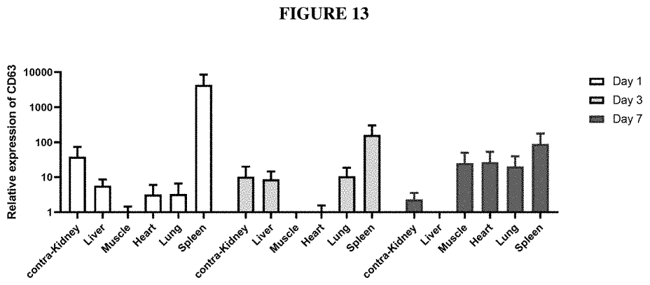

In a fifth set of experiments, induced mesenchymal stem cells (iMSCs) were electroporated with an mRNA construct encoding either Green Fluorescent Protein (GFP) or CD63-GFP by standard methods as described herein. Electroporated iMSCs were injected under the capsule of one kidney in immunocompromised mice, with the contralateral kidney serving as an uninjected control. Local administration under the kidney capsule area retained the injected cells and ensured that delivery of mRNA in vivo was through a cell-independent manner. Samples were collected from the treated and untreated kidney, as well as liver, spleen, lung, muscle, and heart tissue for analysis. Quantitative PCR was performed on extracted RNA to detect expression of exogenous CD63 mRNA, which does not exist naturally, indicative of the distribution of the mRNA cargo in vivo. As shown in FIG. 13, the exogenous CD63 mRNA could be detected in every organ tested at least at some point over the course of 7 days, and in many tissues over the entire 7 days. This broad distribution of CD63 mRNA expression indicates a systemic delivery of the mRNA cargo in vivo, despite the mRNA-loaded cells being administrated locally (i.e., under the kidney capsule). Immunofluorescent staining using anti-GFP antibody confirmed expression of GFP (translated from either the CD63-GFP or GFP mRNA construct) in kidney and spleen tissue (data not shown).

In summary, these experiments demonstrate that mRNA agents can be efficiently loaded into stem cells in vitro and that protein expression from the mRNA agent in vitro and in vivo generally is greater using electroporation for mRNA loading as compared to using LNP-mediated transfection for mRNA loading. Moreover, the experiments demonstrate that mRNA agents in a delivery vehicle that is administered locally in vivo, such as under the kidney capsule, can nevertheless exhibit systemic expression of the mRNA, including broad organ distribution.

### Example 2: Use of Extroducer for Delivery of Stem Cells In Vivo

In this example, human mesenchymal stem cells (MSCs) were delivered into swine heart tissue in vivo using the catheter-type delivery system referred to herein as the Extroducer.

In a first set of experiments, the effect of passaging human MSCs through the Extroducer in vitro was examined. The MSCs were first modified by electroporation with modified mRNA encoding green fluorescent protein (GFP)(5 μg) and modified mRNA encoding vascular endothelial growth factor (VEGF)(5 μg) and cryopreserved. Following thawing, the modified MSCs were either immediately seeded on culture vessels or were passed through the Extroducer before being seeded. 1×10⁶ cells in 100 μl media were passaged through the Extroducer. Test #1, in which the cells were passed through the Extroducer for 3 minutes and 47 seconds, resulted in 72% cell recovery and 72% cell viability. Test #2, in which cells were passed through the Extroducer for 3 minutes and 10 seconds, resulted in 77% cell recovery and 75% cell viability. Since the thawed MSCs that were immediately plated exhibited 75% viability, the results from this first set of experiments indicated that passage of the MSCs through the Extroducer did not significantly affect cell viability.

Next, the engraftment of the MSCs into swine heart tissue following in vivo delivery using the Extroducer was examined. Healthy naïve pigs, with no immunosuppression, were used as the recipients. To evaluate the retention of the MSCs in the swine heart, MSCs radiolabeled with Zr89 were delivered into the cardiac apex of healthy pigs using the Extroducer (n=3). For comparison, radiolabeled MSCs were also delivered into the cardiac apex of healthy pigs using a 26 gauge needle (n=3). All animals were followed for five days post-injection. Gamma counter measurements were performed to determine the % retention of the injected dose (ID). The results are summarized below in Table 1, with radioactivity measured in Megabecquerel (MBq):

TABLE 1

| | | Gamma Counter | | |
|---|---|---|---|---|
| | | Injected Dose | | |
| Animal | Device | (MBq) | MBq | % ID |
| 1 | Extroducer | 0.7 | 0.3 | 44% |
| 2 | | 1.2 | 0.4 | 38% |
| 3 | | 1.3 | 0.2 | 16% |
| 4 | 26 Gauge | 4.1 | 0.2 | 6% |
| 5 | Needle | 1.4 | 0.0 | 0% |
| 6 | | 1.2 | 0.1 | 5% |

The results demonstrate that the Extroducer was significantly better than the 26 gauge needle at delivering the MSCs to the cardiac apex in swine hearts, with cell retention being at least 3-fold higher, and as much as 8- to 9-fold higher, in animals treated with the Extroducer versus the 26 gauge needle. Moreover, none of the animals treated with the 26 gauge needle achieved even 10% retention of the injected dose of radiolabeled cells, whereas all of the animals treated with the Extroducer exhibited more than 10% retention of the injected dose of radiolabeled cells. Individual Extroducer-treated animals exhibited greater than 15%, greater than 35% and greater than 40% retention of the injected dose of radiolabeled cells, thereby demonstrating that the Extroducer is capable of delivering cells into the heart such that a significant portion of the delivered cells are retained within the heart.

### Example 3: Release of Tetraspanin-Positive Exosomes by iMSCs

In this example, the expression of tetraspanins by iMSCs, and the release of tetraspanin-positive exosomes from the iMSCs, was examined.

In a first set of experiments, induced MSCs (iMSCs) were prepared and expression of tetraspanin (TSPAN) proteins was examined by standard methods using anti-TSPAN antibodies. The experiments demonstrated that the iMSCs expressed the TSPAN proteins CD9, CD63 and CD81. Additionally, tetraspanin-positive exosomes could be visualized with tomographic imaging.

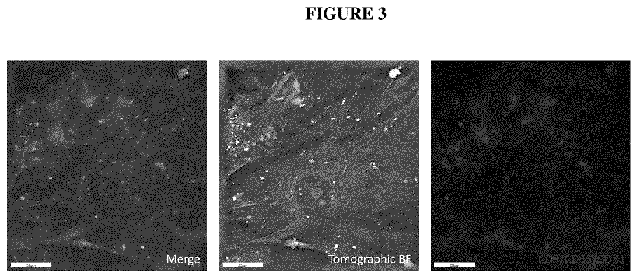

Representative images showing that iMSC TSPAN markers CD9, CD63, CD81 are localized on tomographic bright-field visible intracellular/extracellular vesicles, are shown in FIG. 3.

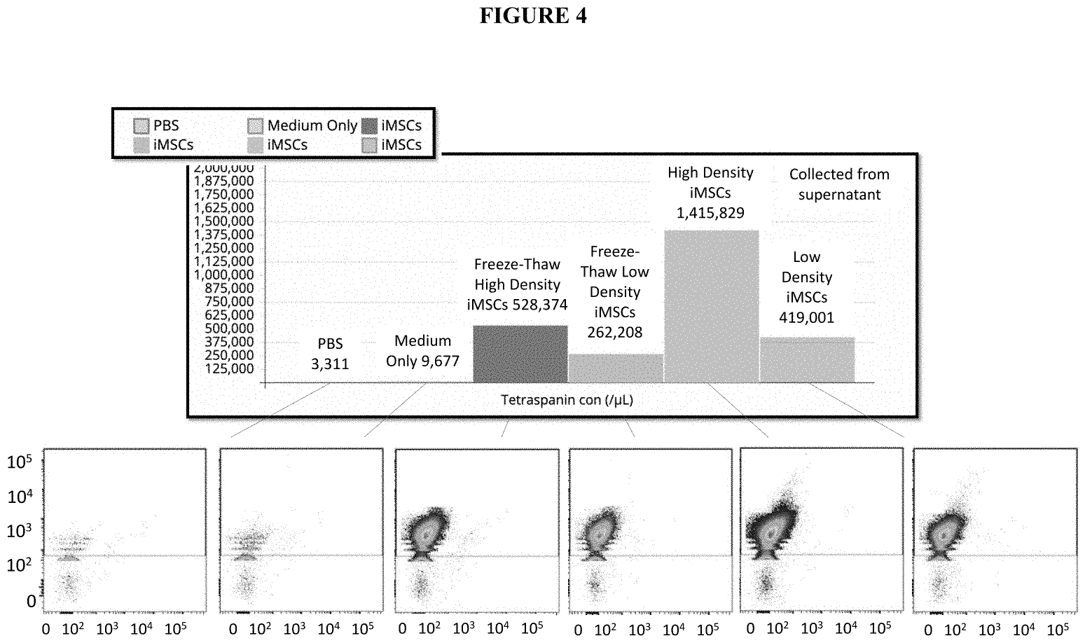

Next, the release of TSPAN-positive exosomes from the iMSCs was examined. The tetraspanin concentration in the supernatants of high density iMSCs and low density iMSCs was determined, as well as the concentration in the supernatants after freeze-thawing of the high- or low-density iMSCs. The results are shown in FIG. 4, with PBS and medium only controls. The results demonstrate that the iMSCs release TSPAN-positive exosomes into the supernatant, with the high density iMSCs that had not undergone freeze-thawing exhibiting the highest concentration in the supernatants.

Thus, this example demonstrates that iMSCs express TSPAN proteins and release TSPAN-positive exosomes into the supernatant upon cell culture.

### Example 4: Loading of iMSCs and Exosomes with mRNA Agents

In this example, a variety of approaches were used to load iMSCs and/or exosomes with mRNA agents.

In a first set of experiments, iMSCs were transfected with a labeled mRNA construct and the presence of labeled mRNA was detected in intracellular vesicles of the iMSCs post-transfection. Furthermore, upon further culture of the transfected cells, labeled mRNA was detected outside of the cells in extracellular vesicles that were released from the transfected iMSCs. Thus, extracellular vesicles containing the mRNA agent were obtainable by transfecting the iMSCs with the mRNA.

Fluorescent nucleotide modified mRNA-594 encoding the fluorescent protein GFP (mRNA-594-GFP) was loaded into pluripotent stem cell derived mesenchymal stem cells (iMSCs) either by electroporation or by transfection using the Lipofectamine™ RNAiMAX reagent (ThermoFisher Scientific), an LNP reagent. The mRNA-594-GFP dosage used was 5 ug per one million iMSCs.

Electroporation of mRNA-594-GFP into iMSCs was performed using the Lonza Nucleofector 2b device. The mRNA-594-GFP loading into iMSCs via electroporation was performed as follows: The Supplemented Nucleofector solution was pre-warmed at room temperature while mRNAs were maintained on ice up until transfection mix was made. 70-80% confluent iMSC cultures were aspirated and washed once with PBS prior to enzymatic disassociation using TrypLE (ThermoFisher Scientific) for 5-7 minutes. Disassociated cells were washed from the well using mild trituration of added basal medium at 1:1 proportion (1 ml TrypLE: add 1 ml basal media). Suspension is spun down at 300 g or RCF for 5 min and supernatant is discarded. Cell pellet is resuspended in a 15 ml conical at a ratio of 1e\hat{ }6 cells per mL. To perform nucleofection: Following centrifugation of aliquoted suspension, supernatant was carefully and completely removed before the cells were resuspended in RT Nucleofector Solution (Lonza Nucleofector kit) at a ratio of 100 uL per 1e\hat{ }6 cells. The Nucleofector solution contained 82 uL Solution A (Tube A from the kit), 18 uL Solution B (Tube B from the kit) and desired amount of mRNA-594-GFP.

It is important to minimize cell-mRNA contact with the Nucleofection solution for over 15 mins, due to the negative influence on cell viability and mRNA integrity. The cell—Nucleofection solution was transferred into a cuvette (supplied with Lonza's nucleofection kit), with minimal air bubbles and the selected program (C-017 or U-020) was applied. Immediately after nucleofection, 500 uL of pre-equilibrated iMSC culture media was added to each electroporated sample, and cells were plated onto Ibidi 35 mm imaging dish (Ibidi Cat #88156). Cells were left to equilibrate and reattach at 37° C. for four hours. Following nucleofection and recovery, 35 mm Ibidi dish was moved to Nanolive 3D-Cell Explorer-Fluo for imaging. Nanolive stage incubator and gas composition were maintained at 37° C. and normoxia for all tomographic imaging experiments.

Alternatively, transfection of iMSCs with mRNA-594-GFP was performed using RNAiMAX, according to the manufacturer's instructions. Expression of 594 fluorescence with simultaneous tomographic imaging in iMSCs was also performed using the Nanolive 3D-Cell Explorer-Fluo.

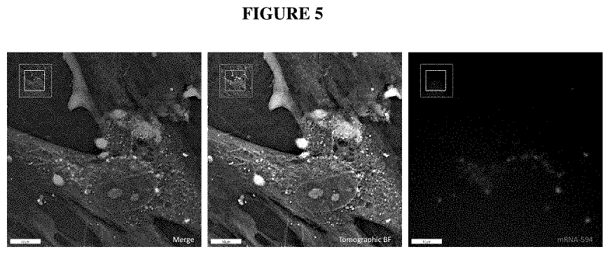

Representative images using the Nanolive 3D Cell Explorer-Fluo to visualize transfected mRNA-594-GFP contained in tomographic bright-field visible vesicles within intracellular compartments are shown in FIG. 5.

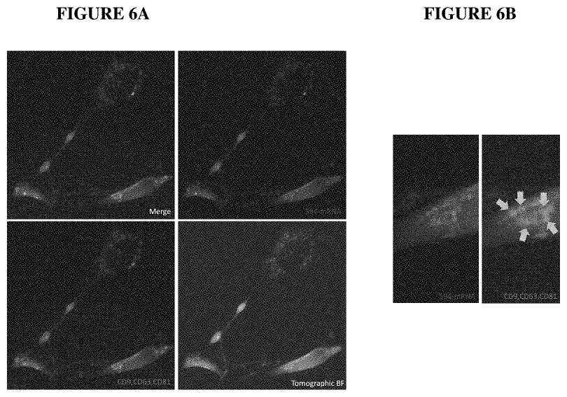

Representative images using the Nanolive 3D Cell Explorer-Fluo to visualize transfected mRNA-594-GFP contained in tomographic bright-field visible vesicles, that are also TSPAN (CD9/63/81) positive within intracellular compartments, are shown in FIG. 6A-6B.

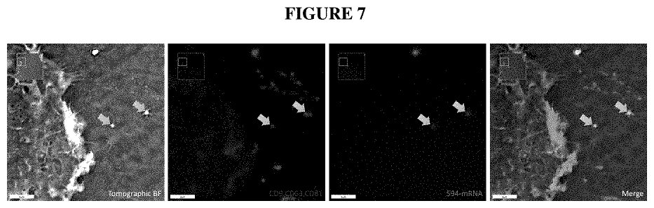

Representative images using the Nanolive 3D Cell Explorer-Fluo to visualize transfected mRNA-594-GFP contained in tomographic bright-field visible vesicles, that are also TSPAN (CD9/63/81) positive within extracellular compartments, are shown in FIG. 7.

In a second set of experiments, labeled mRNA construct was transfected into iMSCs and the presence of the mRNA protein product in exosomes derived from the iMSCs was detected. The results demonstrated that the exosomes derived from the mRNA-transfected iMSCs did contain detectable mRNA protein product.

mmRNA-GFP encoding the fluorescent protein GFP (mRNA-GFP) was loaded into pluripotent stem cell derived mesenchymal stem cells (iMSCs) either by electroporation or by transfection using the Lipofectamine™ RNAiMAX reagent (ThermoFisher Scientific), an LNP reagent. The mRNA-GFP dosage used was 5 ug per one million iMSCs.

Electroporation of mRNA-GFP into iMSCs was performed using the Lonza Nucleofector 2b device. The mRNA-GFP loading into iMSCs via electroporation was performed as follows: The Supplemented Nucleofector solution was pre-warmed at room temperature while mRNAs were maintained on ice up until transfection mix was made. 70-80% confluent iMSC cultures were aspirated and washed once with PBS prior to enzymatic disassociation using TrypLE (ThermoFisher Scientific) for 5-7 minutes. Disassociated cells were washed from the well using mild trituration of added basal medium at 1:1 proportion (1 ml TrypLE: add 1 ml basal media). Suspension is spun down at 300 g or RCF for 5 min and supernatant is discarded. Cell pellet is resuspended in a 15 ml conical at a ratio of 1e\hat{ }6 cells per mL. To perform nucleofection: Following centrifugation of aliquoted suspension, supernatant was carefully and completely removed before the cells were resuspended in RT Nucleofector Solution (Lonza Nucleofector kit) at a ratio of 100 uL per 1e\hat{ }6 cells. The Nucleofector solution contained 82 uL Solution A (Tube A from the kit), 18 uL Solution B (Tube B from the kit) and desired amount of mRNA-GFP.

It is important to minimize cell-mRNA contact with the Nucleofection solution for over 15 mins, due to the negative influence on cell viability and mRNA integrity. The cell—Nucleofection solution was transferred into a cuvette (supplied with Lonza's nucleofection kit), with minimal air bubbles and the selected program (C-017 or U-020) was applied. Immediately after nucleofection, 500 uL of pre-equilibrated iMSC culture media was added to each electroporated sample, and cells were plated onto Ibidi 35 mm imaging dish (Ibidi Cat #88156). Cells were left to equilibrate and reattach at 37° C. for four hours. Following nucleofection and recovery, 35 mm Ibidi dish was moved to Nanolive 3D-Cell Explorer-Fluo for imaging. Nanolive stage incubator and gas composition were maintained at 37° C. and normoxia for all tomographic imaging experiments.

Alternatively, transfection of iMSCs with mRNA-GFP was performed using RNAiMAX, according to the manufacturer's instructions. Expression of GFP fluorescence with simultaneous tomographic imaging in iMSCs was also performed using the Nanolive 3D-Cell Explorer-Fluo.

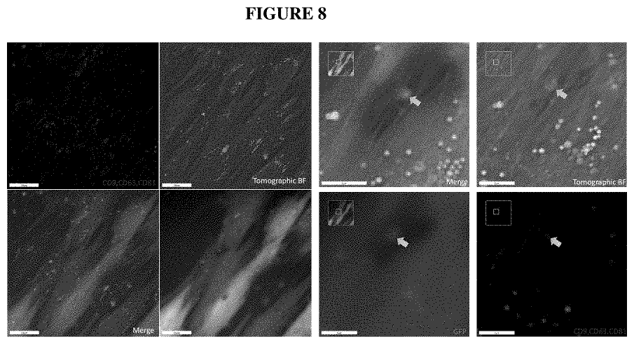

Representative images using the Nanolive 3D Cell Explorer-Fluo to visualize translated GFP protein contained in tomographic bright-field visible vesicles, that are also TSPAN (CD9/63/81) positive within intracellular/extracellular compartments, are shown in FIG. 8.

These results demonstrate that the exosomes derived from the mRNA-transfected (LNP/electroporation) iMSCs did contain detectable mRNA protein product.

### Example 5: Delivery of iMSC-Derived Exosomes to Beating Cardiomyocytes

In this example, iMSC-derived exosomes were used to deliver an mRNA cargo to beating cardiomyocytes in culture.

mmRNA-GFP encoding the fluorescent protein GFP (mRNA-GFP) was loaded into pluripotent stem cell derived mesenchymal stem cells (iMSCs) via transfection of mRNA-GFP performed using RNAiMAX, according to the manufacturer's instructions. iMSCs were transfected with 10 ug per 1e\hat{ }6 cells. Media was changed 4 hours after transfection, 2 ml per sample.

Isolation of iMSC EVs: From 80% confluent 6-well plate (approximately 2 million cells), 12 ml iMSC supernatant was collected 28 hours after transfection. Per the Izon EV isolation protocol, was spun down once at 200 g for 10 min, moved supernatant to a new tube and spun at 2000 g for 10 min. Supernatant was then carefully removed and concentrated using Amicon Filter Units (MWCO=100 kDa; Merck Millipore) until final volume of 150 ul is reached. Using Izon Automatic Fraction Collector (AFC1) mounted with Izon qEV Single Column, concentrated input volume was overlayed on the column. 1 ml of buffer was eluted and discarded before sample elution of 600 uL exosome isolate that were fractioned into 4×150 ul aliquots based on size proportion using the AFC1. The complete isolate was retained for downstream application and the four fractions were combined. 600 uL complete isolate was further concentrated to a volume of 15 uL using Amicon Filter Unit (MWCO=100 kDa; Merck Millipore) prior to its administration to cardiomyocytes.

Cardiomyocyte differentiation from pluripotent stem cells was completed after 14 days of using the established protocol from Foo et al., 2018 Molecular Therapy. iMSC Exosome isolate was added to beating cardiomyocytes at day 15 and were imaged on day 16.

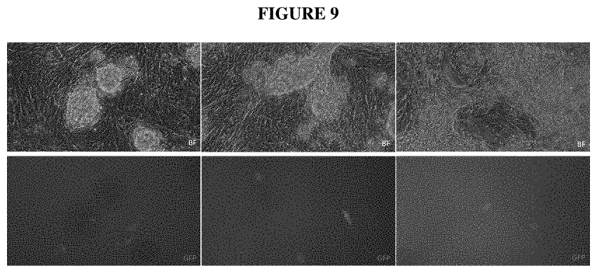

Representative images using standard bright-field and fluorescence microscopy to visualize translated GFP protein expressed in beating cardiomyocytes are shown in FIG. 9.

The results demonstrated that isolated exosomes from iMSCs could be successfully delivered into beating cardiomyocytes, delivering their payload of either mRNA or mRNA protein product.

### Example 6: Modification of iMSCs with TSPAN to Enhance Exosome Formation

In this example, the iMSCs were modified to express exogenously-introduced TSPAN proteins and the effect on exosome formation was examined.

mRNA constructs encoding labeled TSPAN proteins were designed and prepared using green fluorescent protein (GFP) as the label. Constructs encoding CD9-GFP, CD63-GFP and CD81-GFP were used (5 μg each). The labeled TSPAN constructs were introduced into iMSCs by both LNP transfection and electroporation using the methods previously described in Example 4. iMSCs transfected with labeled TSPAN construct were plated on ibidi microscopy dishes and imaged using the Nanolive 3D Cell Explorer-Fluo as described in Example 4. Expression of the labeled TSPAN proteins was detectable in the iMSCs at 1 hour and 6 hours post-transfection, evident by its displacement of the TSPAN antibody there is an observable increase of labeled vesicle. By 12 hours post-transfection, nearly all of the tomographically visible exosomes were tagged with the labeled TSPAN proteins and abundance of TSPAN was observably higher than endogenous expression.

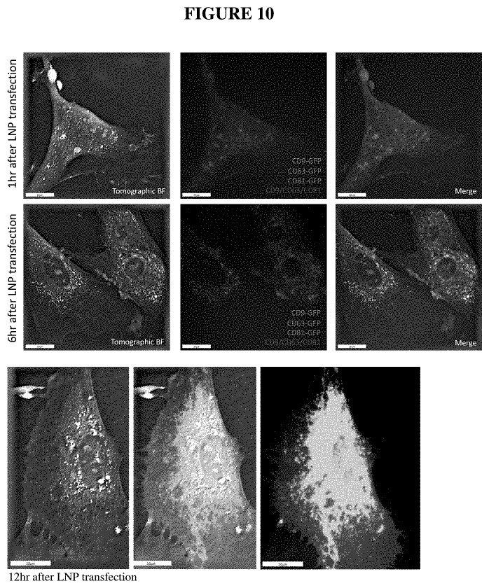

Representative images using the Nanolive 3D Cell Explorer-Fluo to visualize translated TSPAN-GFP protein contained in tomographic bright-field visible vesicles, while also using TSPAN (CD9/63/81) conjugated antibodies to visualize total TSPAN, are shown in FIG. 10.

These results demonstrate that modification of the iMSCs to express exogenously induced TSPAN proteins led to enhancement of exosomes that carried the introduced TSPAN proteins.

### Example 7: Delivery of Large mRNAs Using iMSC-Derived Extracellular Vesicles

In this example, extracellular vesicles (EVs) of iMSCs were loaded with large mRNAs encoding functional macro-molecules and used to deliver the mRNA into target cells.

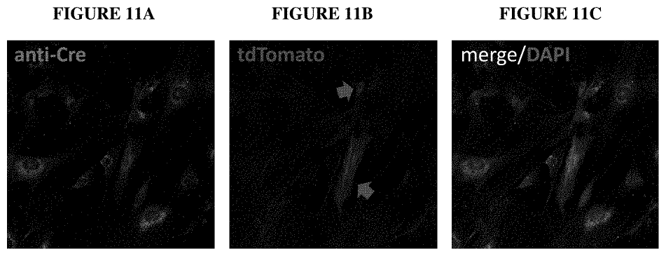

In a first set of experiments, the Cre-LoxP system was used to test the ability to deliver functional macro-molecules using iMSC EVs. In the system, which uses the tdTomato report line, Cre recombinase remodels LoxP loci and initiates the expression of tdTomato fluorescent protein. iMSCs were loaded with Cre recombinase mRNA (10 ug per million iMSC) through electroporation as described in previous examples. 24 hours after electroporation, supernatant from iMSC culture medium was collected, to thereby obtain mRNA-loaded EVs, and used to treat mouse ROSA26:tdTomato reporter cell line. Two days later, Cre recombinase was detected in mouse cells (see FIG. 11A) by using immunofluorescent staining with anti-Cre antibody (mouse cell does not have endogenous Cre expression). tdTomato-positive mouse cells could be observed as well (see FIG. 11B), indicating the mRNA-encoded Cre recombinase was functional in inducing the expression of tdTomato in mouse cells.

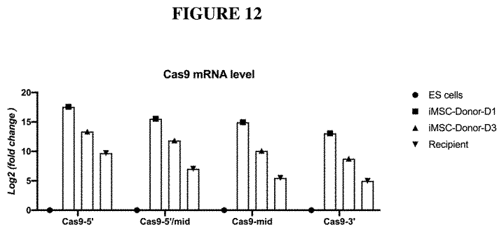

In a second set of experiments, the CRISPR-Cas9 system was used to test the ability to deliver a large mRNA encoding a functional macro-molecule using iMSC EVs. iMSCs were loaded with Cas9 mRNA (4.2 kb) through electroporation as described in previous examples. Levels of Cas9 mRNA levels were determined in two donor cells (iMSC-Donor-D1 and iMSC-Donor-D3) and in recipient cells (Recipient). Representative results are shown in FIG. 12. The results demonstrated that large amounts of Cas9 mRNA was detectable in both iMSC donor cells and in recipient cells, indicating that the iMSC EVs successfully delivered a large mRNA from donor to recipient cells. Additionally, examination of the duration of Cas9 mRNA expression in donor cells demonstrated that mRNA could still be detected three days after electroporation in one of the donor cells, indicating the possibility of using this system for chronic treatment in vivo.

### INCORPORATION BY REFERENCE

Each patent, publication, and non-patent literature cited in the application is hereby incorporated by reference in its entirety as if each was incorporated by reference individually.

1. A method of delivering an mRNA agent to a subject, the method comprising administering to the subject a composition comprising extracellular vesicles (EVs) prepared from human stem cells or human progenitor cells, wherein the EVs encapsulate the mRNA agent.

2. The method of claim 1, wherein the mRNA agent comprises at least one modified nucleotide base.

3. The method of claim 1, wherein the mRNA agent comprises all unmodified nucleotide bases.

4. The method of claim 1, wherein the EVs are exosomes prepared from human mesenchymal stem cells (MSCs).

5. The method of claim 1, wherein the EVs are exosomes prepared from stem cells or progenitor cells selected from the group consisting of human embryonic stem (ES) cells, human induced pluripotent stem cells (iPSCs), human induced mesenchymal stem cells (iMSCs), human cardiac progenitor cells and human pancreatic progenitor cells.

6. The method of claim 1, wherein the stem cells or progenitor cells are hypo-immunogenic.

7. The method of claim 1, wherein the mRNA agent encodes an enzyme, cytokine or growth factor.

8. The method of claim 1, wherein the mRNA agent encodes an antigen or antibody.

9. The method of claim 1, wherein the mRNA agent encodes an immunomodulatory agent.

10. The method of claim 1, wherein the composition is administered to an intraorgan site in the subject.

11. The method of claim 10, wherein the intraorgan site is within the heart.

12. The method of claim 10, wherein the intraorgan site is within the kidney, the pancreas, the liver, the lungs or the brain.

13. The method of claim 1, wherein the mRNA agent is encapsulated in the EVs by introducing the mRNA agent into the human stem cells or human progenitor cells and preparing EVs from the human stem cells or progenitor cells to thereby encapsulate the mRNA agent in the EVs.

14. A method of expressing a protein in a cell, the method comprising:
encapsulating an mRNA agent encoding the protein in extracellular vesicles (EVs) prepared from human stem cells or human progenitor cells, wherein the mRNA agent is at least 300 bases in length; and
transfecting the cells with the EVs such that the mRNA agent expresses the protein in the cell.

15. The method of claim 14, wherein the mRNA agent is at least 1 kb in length.

16. The method of claim 14, wherein the EVs are exosomes.

17. The method of claim 16, wherein the exosomes are prepared from human mesenchymal stem cells (MSCs).

18. The method of claim 17, wherein the MSCs are induced MSCs (iMSCs).

19. The method of claim 14, wherein the mRNA agent encodes an enzyme, a cytokine, a growth factor, an antigen, an antibody or an immunomodulatory protein.

20. The method of claim 14, wherein the protein is Cre recombinase, CRISPR Cas 9 protein, VEGF or phospholamban (PLN).

21. The method of claim 14, wherein the EVs are administered to a subject to thereby deliver the protein to cells of the subject in vivo.

22. The method of claim 21, wherein the EVs are administered to an intraorgan site in the subject.

23. The method of claim 22, wherein the intraorgan site is within the heart.

24. The method of claim 22, wherein the intraorgan site is within the kidney, the pancreas, the liver, the lungs or the brain.

25. The method of claim 21, wherein the EVs are administered to an extravascular site in the subject.

26. The method of claim 21, wherein the EVs are administered using an endoluminal delivery device.

27. A composition comprising exosomes prepared from human induced mesenchymal stem cells (iMSCs), wherein the exosomes encapsulate an mRNA agent at least 300 bases in length.

28. The composition of claim 27, wherein the mRNA agent comprises at least one modified nucleotide base.

29. The composition of claim 27, wherein the mRNA agent comprises all unmodified nucleotide bases.

30. The composition of claim 27, wherein the mRNA agent is at least 1 kilobase in length.

31. The composition of claim 27, wherein the mRNA agent encodes an enzyme, a cytokine, a growth factor, an antigen, an antibody or an immunomodulatory protein.

32. The composition of claim 27, wherein the mRNA agent encodes Cre recombinase, CRISPR Cas 9 protein, VEGF or phospholamban (PLN).
